(19) United States
(12) Patent Application Publication
(10) Pub. No.: US 2005/0107687 A1
(43) Pub. Date: May 19, 2005

(54) System and method for distortion reduction in an electromagnetic tracker

(76) Inventor: Peter Traneus Anderson, Andover, MA (US)

Correspondence Address: MCANDREWS HELD & MALLOY, LTD, 500 WEST MADISON STREET, SUITE 3400, CHICAGO, IL, 60661, US

(21) Appl. No.: 10/869,594

(22) Filed: Jun. 16, 2004

Related U.S. Application Data
(60) Provisional application No. 60/520,138, filed on Nov. 14, 2003.

(51) Int. Cl. A61B 5/05

(52) U.S. Cl. 600/424; 702/127; 702/85; 324/200; 324/207.12

(57) ABSTRACT

Certain embodiments of the present invention provide a system and method for distortion analysis and reduction in an electromagnetic tracker. Certain embodiments of the system include a tracking analysis unit for analyzing a tracking behavior of an instrument and a tracking modification unit for compensating for the tracking behavior of the instrument. The tracking modification unit may compensate for the tracking behavior of the instrument by adjusting the tracking behavior of the instrument. The tracking modification unit may also compensate for the tracking behavior of the instrument by adjusting a tracking system for tracking the instrument. The tracking analysis unit may test the adjusted tracking behavior of the instrument. In an embodiment, the tracking analysis unit generates a map and/or a model of a distortion characteristic of the instrument. The distortion characteristic may be a magnetic field. The distortion handling system is a computer simulated distortion handling system.

### RELATED APPLICATIONS

The present application relates to, and claims priority from, U.S. Provisional Application No. 60/520,138 filed on Nov. 12, 2003, and entitled “System and Method for Distortion Reduction in an Electromagnetic Tracker” (Attorney Docket Number 1333165NV).

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to an electromagnetic tracking system. In particular, the present invention relates to a system and method for reducing distortion caused by tools and other elements in an electromagnetic tracking system.

Medical practitioners, such as doctors, surgeons, and other medical professionals, often rely upon technology when performing a medical procedure, such as image-guided surgery or examination. A tracking system may provide positioning information for the medical instrument with respect to the patient or a reference coordinate system, for example. A medical practitioner may refer to the tracking system to ascertain the position of the medical instrument when the instrument is not within the practitioner's line of sight. A tracking system may also aid in pre-surgical planning.

The tracking or navigation system allows the medical practitioner to visualize the patient's anatomy and track the position and orientation of the instrument. The medical practitioner may use the tracking system to determine when the instrument is positioned in a desired location. The medical practitioner may locate and operate on a desired or injured area while avoiding other structures. Increased precision in locating medical instruments within a patient may provide for a less invasive medical procedure by facilitating improved control over smaller instruments having less impact on the patient. Improved control and precision with smaller, more refined instruments may also reduce risks associated with more invasive procedures such as open surgery.

Tracking systems may also be used to track the position of items other than medical instruments in a variety of applications. That is, a tracking system may be used in other settings where the position of an instrument in an object or an environment is difficult to accurately determine by visual inspection. For example, tracking technology may be used in forensic or security applications. Retail stores may use tracking technology to prevent theft of merchandise. In such cases, a passive transponder may be located on the merchandise. A transmitter may be strategically located within the retail facility. The transmitter emits an excitation signal at a frequency that is designed to produce a response from a transponder. When merchandise carrying a transponder is located within the transmission range of the transmitter, the transponder produces a response signal that is detected by a receiver. The receiver then determines the location of the transponder based upon characteristics of the response signal.

Tracking systems are also often used in virtual reality systems or simulators. Tracking systems may be used to monitor the position of a person in a simulated environment. A transponder or transponders may be located on a person or object. A transmitter emits an excitation signal and a transponder produces a response signal. The response signal is detected by a receiver. The signal emitted by the transponder may then be used to monitor the position of a person or object in a simulated environment.

Tracking systems may be ultrasound, inertial position, or electromagnetic tracking systems, for example. Electromagnetic tracking systems may employ coils as receivers and transmitters. Typically, an electromagnetic tracking system is configured in an industry-standard coil architecture (ISCA). ISCA uses three colocated orthogonal quasi-dipole transmitter coils and three colocated quasi-dipole receiver coils. Other systems may use three large, non-dipole, non-colocated transmitter coils with three colocated quasi-dipole receiver coils. Another tracking system architecture uses an array of six or more transmitter coils spread out in space and one or more quasi-dipole receiver coils. Alternatively, a single quasi-dipole transmitter coil may be used with an array of six or more receivers spread out in space.

The ISCA tracker architecture uses a three-axis dipole coil transmitter and a three-axis dipole coil receiver. Each three-axis transmitter or receiver is built so that the three coils exhibit the same effective area, are oriented orthogonally to one another, and are centered at the same point. If the coils are small enough compared to a distance between the transmitter and receiver, then the coil may exhibit dipole behavior. Magnetic fields generated by the trio of transmitter coils may be detected by the trio of receiver coils. Using three approximately concentrically positioned transmitter coils and three approximately concentrically positioned receiver coils, for example, nine parameter measurements may be obtained. From the nine parameter measurements and one known position or orientation parameter, a position and orientation calculation may determine position and orientation information for each of the transmitter coils with respect to the receiver coil trio with three degrees of freedom.

Many medical procedures involve a medical instrument, such as a drill, a catheter, scalpel, scope, shunt or other tool. Many instruments used in medical activities include metal components. Additionally, an environment surrounding a medical instrument or a tracking system may include metal. Metal or other such substances may distort magnetic fields in the electromagnetic tracking system. Distortions in the electromagnetic tracking system may cause the tracking system to be inaccurate.

Medical practitioners, for example, rely on electromagnetic trackers to perform sensitive image-guided surgery. Accuracy of position measurement is important when guiding a precision instrument in a patient without a direct line of sight. Distortion may produce inaccurate position measurements and potential danger to a patient. Thus, a system that reduces inaccurate tracking measurements would be highly desirable. A system that minimizes the effect of distortion on position measurement would be highly desirable.

Thus, a need exists for a system and method for reducing distortion caused by tools and other elements in an electromagnetic tracking system.

### BRIEF SUMMARY OF THE INVENTION

Certain embodiments of the present invention provide a system and method for distortion analysis and reduction in an electromagnetic tracker. A certain embodiment of an improved electromagnetic tracking system includes a transmitter and receiver for tracking an object and a distortion handling system for analyzing a distortion characteristic of the object. The transmitter transmits a signal, and the receiver receives the signal from the transmitter.

In an embodiment, the distortion handling system analyzes the signal received by the receiver to determine tracking accuracy. The distortion handling system may include a virtual electromagnetic tracker for simulating an effect of the distortion characteristic on tracking. The distortion characteristic may be a magnetic field. The distortion handling system may adjust a tracking behavior of the object.

A certain embodiment of a distortion handling system includes a tracking analysis unit for analyzing a tracking behavior of an instrument and a tracking modification unit for compensating for the tracking behavior of the instrument. The tracking modification unit may compensate for the tracking behavior of the instrument by adjusting the tracking behavior of the instrument. The tracking modification unit may also compensate for the tracking behavior of the instrument by adjusting a tracking system for tracking the instrument. The tracking analysis unit may test the adjusted tracking behavior of the instrument. In an embodiment, the tracking analysis unit generates a map and/or a model of a distortion characteristic of the instrument. The distortion characteristic may be a magnetic field. In an embodiment, the distortion handling system is a computer simulated distortion handling system.

A certain embodiment of a virtual tracking system includes an object simulation module for simulating an object to be tracked and a simulation toolset for analyzing at least one distortion characteristic of the object. The simulation toolset is capable of generating distortion information for the object based on the distortion characteristic(s). In an embodiment, the simulation toolset includes an accuracy module for determining accuracy of simulated tracking of the object. The simulation toolset may include a distortion detection module for determining an impact of distortion from the object on tracking accuracy. The simulation toolset may also include a distortion modeling module for generating an electromagnetic model for evaluating a distorting effect of the object on a tracker environment. Additionally, the simulation toolset may include a distortion compensation module for improving distortion tolerance of an electromagnetic tracker.

A certain embodiment of a method for distortion analysis in an electromagnetic tracking system includes measuring a tracking behavior of an object and analyzing the tracking behavior of the object to determine a distortion effect. The analysis may further include generating a magnetic field map and/or a model of the object to analyze the tracking behavior of the object to determine the distortion effect. The method may also include simulating tracking of the object to determine the distortion effect. Additionally, the method may include modeling electromagnetic fields in an electromagnetic tracking environment to analyze the distortion effect. The method may also include improving distortion tolerance in an electromagnetic tracking system based on the distortion effect.

In an embodiment, the method includes adjusting the tracking behavior of the instrument to reduce the distortion effect. The method may further include testing the instrument to verify a reduced distortion effect. In an embodiment, the method includes adjusting an electromagnetic tracker to compensate for the distortion effect. The method may further include testing the instrument to verify a reduced distortion effect.

### BRIEF DESCRIPTION OF SEVERAL VIEWS OF THE DRAWINGS

FIG. 1 illustrates a distortion handling system for use in improving position measurement accuracy in an electromagnetic tracker used in accordance with an embodiment of the present invention.

FIG. 2 illustrates a flow diagram for a method for distortion handling in an electromagnetic tracking system used in accordance with an embodiment of the present invention.

FIG. 3 shows a flow diagram for a method for electromagnetic assessment of an instrument in accordance with an embodiment of the present invention.

FIG. 4 illustrates a virtual tracker for EM tracker development used in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

For the purpose of illustration only, the following detailed description references an embodiment of an electromagnetic tracking system used with an image-guided surgery system. It is understood that the present invention may be used with other imaging systems and other applications.

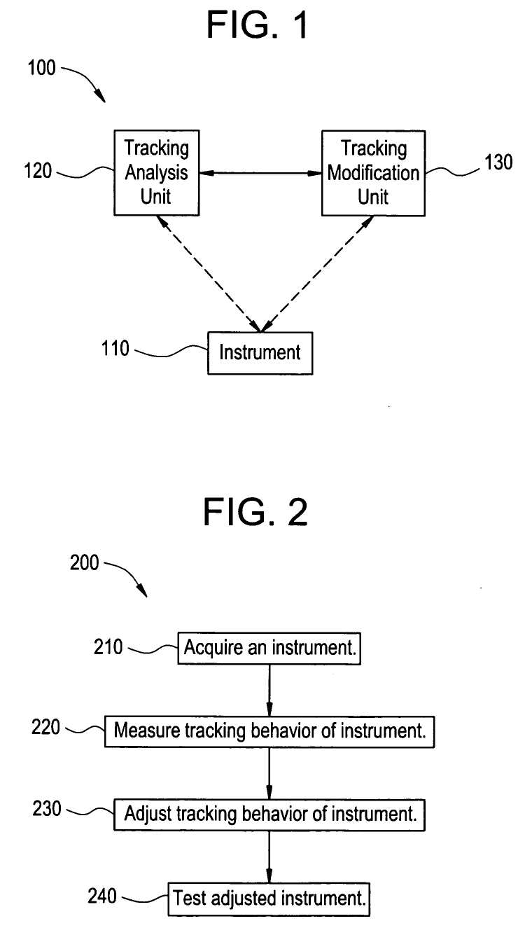

FIG. 1 illustrates a distortion handling system 100 for use in improving position measurement accuracy in an electromagnetic (EM) tracker used in accordance with an embodiment of the present invention. The system 100 includes an instrument 110, a tracking analysis unit 120, and a tracking modification unit 130. The tracking analysis unit 120 observes the tracking behavior of the instrument 110. The tracking modification unit 130 attempts to improve or compensate for the tracking behavior of the instrument 110 based on information from the tracking analysis unit 120. The tracking analysis unit 120 and the tracking modification unit 130 may be implemented in hardware and/or in software as separate units or a single unit. The tracking analysis unit 120 and/or the tracking modification unit 130 may be integrated with the EM tracker or may be a separate system.

The instrument 110 may be any instrument with use in a medical activity, such as an orthopaedic tool (an electric or pneumatic drill, for example), a catheter, scalpel, scope, stent, or other tool. The instrument 110 may generate or affect a magnetic field that causes distortion in readings of the EM tracker. Positioning of an EM navigation device, such as a receiver or transmitter, on the instrument 110 may impact distortion and/or an effect of the distortion on tracking.

The tracking analysis unit 120 analyzes a tracking behavior and effect of the instrument 110. The tracking analysis unit 120 performs actual and/or simulated EM tracking of the instrument 110 to determine an effect of distortion from the instrument 110 on position and/or orientation calculations. The tracking analysis unit 120 may be a computer simulating effects from the instrument 110 in a tracking system. A receiver positioned on the instrument 110, a transmitter, and/or other sensors may be used to gather information about the instrument 110. The tracking analysis unit 120 obtains magnetic field data for the instrument 110. The tracking analysis unit 120 generates position and orientation data for the instrument 110 in a tracking coordinate system. Other effects on instrument 110 tracking may be measured and/or simulated by the tracking analysis unit 120. The tracking analysis unit 120 may also generate a map and/or a model relating to fields and distortion effects around the instrument 110.

The tracking modification unit 130 adjusts or compensates for tracking behavior of the instrument 110. The tracking modification unit 130 uses the map, model, and/or other data from the tracking analysis unit 120 to minimize distortion effects from the instrument 110. The tracking modification unit 130 may test different receiver and/or transmitter configurations with the instrument 110 to improve the tracking behavior of the instrument 110. The tracking modification unit 130 may modify, recalibrate, or reprogram the EM tracker to offset distortion effects from the instrument 110.

Tracking behavior of the instrument 110 may be adjusted or compensated in a variety of manners. For example, a mathematical model of the instrument 110 and/or a system 100 component may be developed to correct for distortion. For example, a magnetic field model may be developed and corrected to reduce errors in tracking the instrument 110. Alternatively, distortion may be modeled, such as using a ring model of instrument 110 distortion, and errors compensated after tracking measurements. Additionally, the instrument 110 or another system 100 component may be shielded to produce a known distortion which may be compensated for before or after tracker measurement. The EM tracker may be calibrated and/or the position of a transmitter or receiver on the instrument 110 may be adjusted to compensate for or reduce distortion in tracking.

another embodiment, a distorter next to the instrument 110, such as next to a three-coil transmitter or receiver on the instrument, may be compensated for by characterizing the instrument 110 including the distorter. Alternatively, using a one-coil transmitter and a receiver array, for example, allows transmitter gain to be tracked, rather than exactly characterizing the transmitter. Gain tracking may help reduce the effect of distortion in instrument 110 tracking.

In an embodiment, the EM tracker includes a transmitter for transmitting a signal, a receiver for receiving the signal from the transmitter, and tracker electronics for analyzing the signal received by the receiver. The tracker electronics may be configured by software. The tracker electronics determines a position and/or an orientation of the instrument 110 in a tracking coordinate system based on information from a receiver and/or a transmitter. In an embodiment, the receiver is placed on the instrument 110 to determine the position and/or orientation of the instrument 110 in relation to the transmitter. In an alternative embodiment, the transmitter may be placed on the instrument 110 to determine the position and/or orientation of the instrument 110 in relation to the receiver. The EM navigation devices used in the EM tracker may be wired and/or wireless devices, for example (such as a wireless transmitter). A configuration of the EM tracker may be adjusted using information from the distortion handling system 100 to compensate for distortion effects from instruments and operating environment.

In operation, the instrument 110 is tracked using the EM tracker or other EM navigation devices. The instrument 110 may be tracked in a physical EM tracker or a simulation of the instrument 110 may be tracked in a virtual tracking system. The tracking analysis unit 120 measures parameters, such as field, position, and orientation data, relating to the tracking of the instrument 110. The tracking analysis unit 120 generates a distortion model and/or field map for the instrument 110. Then, the tracking modification unit 130 uses the model and/or map and additional data from the tracking analysis unit 120 to minimize distortion effects from the instrument 110. The tracking modification unit 130 may test various receiver configurations and/or placements with the instrument 110 to minimize distortion in the EM tracker. For example, placing a receiver assembly at a certain point on a drill may minimize distortion effects caused by a field from the drill. The tracking modification unit 130 may also reconfigure or recalibrate the EM tracker to account for distortion effects from the instrument 110. For example, the EM tracker may be programmed to anticipate a certain distortion caused by a drill being tracked. Additionally, the tracking modification unit 130 may program the EM tracker to disregard distortion effects from the environment, such as metal in a patient positioning surface, a light, and/or a room structure. The tracking analysis unit 120 may then test the instrument 110 in the EM tracker to verify position accuracy. If testing is satisfactory, then the instrument 110 may be used by a medical practitioner and tracked in the EM tracker.

FIG. 2 illustrates a flow diagram for a method 200 for distortion handling in an electromagnetic tracking system used in accordance with an embodiment of the present invention. First, at step 210, an instrument 110 is acquired. In an embodiment, the instrument 110 may be physically or virtually (an image or electronic representation, for example) acquired.

Then, at step 220, the tracking behavior of the instrument 110 is measured. For example, if the instrument 110 has been physically acquired, magnetic field, position, orientation, and/or other data are measured. If the instrument is virtually represented, then the instrument's 110 effect may be simulated on a computer or virtual tracker, for example. Distortion and/or other characteristics of the instrument 110 during electromagnetic tracking may be observed and/or simulated. A map and/or a model may be generated to analyze magnetic fields and distortion effects around the instrument 110.

Next, at step 230, tracking behavior of the instrument 110 or tracker is adjusted to reduce or compensate for distortion effects from the instrument 110. Errors in tracking of the instrument 110 are analyzed. Various instrument 110 and receiver assembly configurations may be tested to determine which configuration minimizes distortion effects and other errors. Calibrations or configurations of the tracker may be tested to determine which configuration most compensates for distortion and other effects.

Additionally, residual errors remaining after the instrument 110 or tracker has been adjusted may be analyzed. Residual errors may be analyzed to determine whether the residual errors are below a certain threshold. For example, if residual errors are small enough, the residual errors may be ignored. Furthermore, stability of residual errors is analyzed. Residual errors may be analyzed to determine whether the residual errors are reliably below a certain threshold, given noise and other factors, for example. In an embodiment, if residual errors are not reliably below a certain threshold, then the instrument 110 or tracker is further adjusted to reduce residual errors.

Then, at step 240, the adjusted instrument 110 and/or tracker are tested to verify tracking accuracy. In an embodiment, position measurements are re-obtained using the map and/or model generated during step 220 above. If tracking data for the instrument 110 meets certain standards or improvement levels, then the instrument 110 may be used with an electromagnetic tracker.

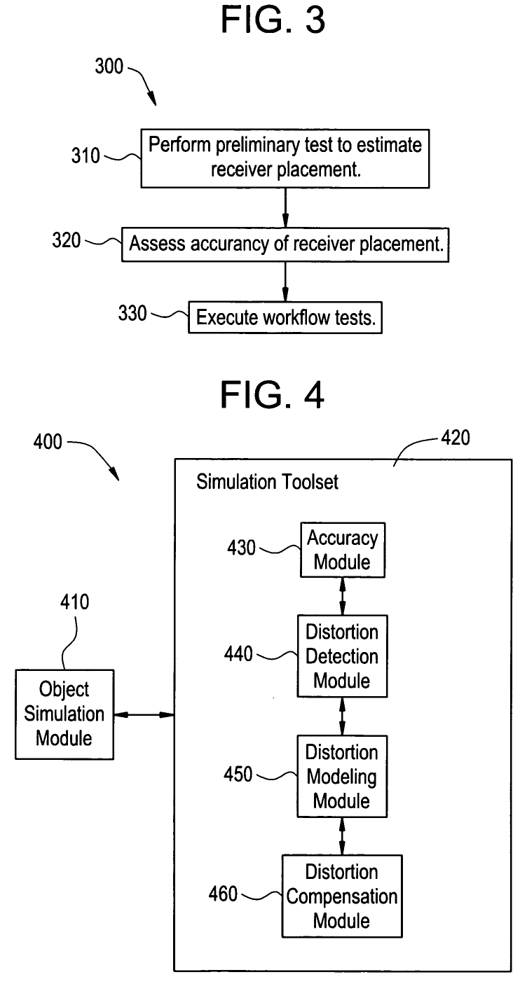

Many methods may be used to assess electromagnetic behavior of the instrument 110. FIG. 3 shows a flow diagram for a method 300 for electromagnetic assessment of an instrument 110 in accordance with an embodiment of the present invention. First, at step 310, a preliminary test may be performed to estimate an optimal receiver assembly placement relative to the instrument 110 being tested. Then, at step 320, receiver placement is assessed through accuracy studies over a working range of values. Next, at step 330, workflow tests are executed to address workflow issues. Workflow tests may be executed with varying levels of detail to determine interaction between the instrument 110 and surgical workflow. Examples illustrating steps of the method 300 will be described further below.

For example, preliminary whiteboard or robot-based testing is used to estimate an optimal EM receiver pack placement relative to the instrument 110 under consideration. An analysis of receiver placement suitability may be done through an analysis of tracking system goodness of fit data for instrument 110 position, for example. Additionally, an analysis of position and orientation data stability over repeated measurement may be performed for fixed EM receiver and transmitter locations.

An EM receiver pack placement may be assessed through robot and manual phantom accuracy analyses. An EM receiver pack may be affixed to the instrument 110 under test and/or a robot assessment fixture. The robot and/or manual phantom may be used to assess relevant error over a working range for the EM tracker for given tip offset lengths. For example, a maximum value, root-mean-squared (rms) value, standard value, and histogram of error may be analyzed for a plurality of measurements. Additionally, a working range of acceptable accuracy may be obtained. An effect of an operating motor on instrument 110 performance may also be analyzed. Furthermore, an ability of an electromagnetic field integrity detector (FID) to detect position errors due to metal distortion of the instrument 110 may be analyzed.

Various workflow tests may be used to address aspects of workflow in the electromagnetic tracker and distortion handling system 100. A range of motion and effects of a patient environment are measured based on out of range locations and FID warnings. In an embodiment, the instrument 110 is represented by a rough approximation. For example, a surgical drill may be represented by a squirt gun. A drill guide may be represented by a wooden dowel with a receiver pack taped on in a likely location, for example. Tests using a rough approximation are intended to provide an initial indication of interaction between instruments and surgical workflow for various applications.

In another embodiment, a test is performed with a closer approximation of the instrument 110. Appropriate materials are used to identify tracking and FID errors. Tests may simulate a patient environment by using a stainless steel sheet and/or bars bolted onto wooden test tables as surrogates for operating room and fracture tables, for example.

another embodiment, a prototype tool may be used to closely imitate the instrument 110 in clinical testing. Concept instrument models may be used in a patient environment, such as a hospital operating room, with cadavers or test subjects, for example. Tracking and FID errors may be identified using the test.

Additionally, a simulation toolset may be used to develop and test EM tracker systems. The simulation toolset may explore tracking performance of different coil architectures, metal tolerance, and/or distortion detection, for example. The toolset may be adjusted to develop and test instrument 110 capability and/or instrument 110 application.

Simulation tools may be used to examine accuracy limitations of an undistorted tracker. A virtual tracker (VT) with a simulation toolset provides a controlled system for examining accuracy limitations due to signal-to-noise ratio, fabrication errors, calibration errors, and other factors. Individual tracker parameters may also be studied for individual and/or combined effect on tracker system accuracy.

Simulation tools may also be used for distortion modeling. In an embodiment, the VT includes electromagnetic modeling of a physical environment. The model may be used to analyze a distorting effect of object in or near a working volume of the tracker. Distorting objects may include surgical tools, room structure (a rebar floor and/or a lead wall, for example), and operating room tables or lights, for example. First, distorting properties of the object(s) are evaluated for a given coil architecture. In an embodiment, distorting properties are evaluated without compensation techniques. The VT is used to derive sensor architectures with improved tolerance to metal distortion, including fixed (tracked surgical instruments, for example) and incidental (tables or clamps, for example) distortion.

The VT may also be used to improve distortion compensation or distortion tolerant tracking. Distortion modeling data may be used to facilitate distortion tolerance in an EM tracking system. For example, passive and/or active shielding techniques allow sensors to be integrated with distorting tools. Distortion mapping techniques, such as position and orientation (P&O) mapping or EM field mapping, may be based on measured data, simulated data, or a combination of data, for example. Extraction of reduced degree of freedom (DOF) parameterized models (multiple dipole models, for example) from measured and/or simulated data may improve distortion tolerance or compensation. In an embodiment, a multi-sensor “mapping chamber” and/or robot data collection may be used to map distortion data.

Furthermore, distortion detection may be improved. FID algorithms may be improved. For example, FID for single position sensors, methods for gathering and analyzing information from multiple sensors, characterizing FID algorithm weaknesses, and model fitting processes may be improved.

FIG. 4 illustrates a virtual tracker (VT) 400 for EM tracker development used in accordance with an embodiment of the present invention. The VT 400 includes an object simulation module 410 and a simulation toolset 420. The VT 400 may be implemented in software on a general purpose computer or a dedicated processor or circuit, for example. The VT 400 maybe integrated with the distortion handling system 100 described above.

The object simulation module 410 simulates an object or instrument 110 to be tracked. Data characteristic of the instrument 110 is generated by the object simulation module 410. The simulation toolset 420 uses the simulation data from the object simulation module 410 to analyze characteristics and tracking behavior of the instrument 110 in a virtual tracking system. In an embodiment, the simulation toolset 420 includes an accuracy module 430, a distortion detection module 440, a distortion modeling module 450, and a distortion compensation module 460.

The accuracy module 430 analyzes the instrument 110 or a virtual representation of the instrument 110 to determine accuracy of a simulated EM tracker and an effect of the instrument 110 on EM tracker accuracy. The accuracy module 430 examines a signal-to-noise ratio, errors such as fabrication errors and calibration errors for the instrument 110, and other factors to determine individual and/or combined effects on tracker measurement accuracy.

The distortion detection module 440 determines an impact of distortion on tracking accuracy. The distortion detection module 440 utilizes field integrity detection (FID), distortion and instrument models, and sensor information to determine a distortion field from the instrument 110. Distortion information from the distortion detection module 440 may be used to adjust the tracker and tracker software to improve tracking accuracy.

The distortion modeling module 450 generates an electromagnetic model of an EM tracker environment. The model is used to evaluate a distorting effect of objects in or near a working volume of the tracker. The distortion modeling module 450 may model distortion fields from a variety of objects, such as the instrument 110 and the environment in which the tracker is operating (walls, floors, patient table, etc.). Information from the distortion modeling module 450 may be used to modify a configuration of the instrument 110 and/or develop tracker sensor architectures with improved tolerance to fixed and/or incidental metal distortion.

The distortion compensation module 460 is used to improve distortion tolerance of the EM tracker and associated systems. The distortion compensation module 460 may be used to simulate an effect of sensor shielding techniques (passive and/or active, for example), distortion mapping techniques, extraction of parameterized object models, and/or field mapping, for example. Simulated results and data may be used to program the EM tracker and/or improve distortion tolerance of the EM tracker.

In certain embodiments, the VT 400 may be used to help provide a tracking system that is robust to incidental distorters. Information from the VT 400 may help to allow tight or ergonomic integration of sensors with surgical tools. Information from the VT 400 may help configure and calibrate instruments, instrument guides, and tracking systems. The modules of the VT 400 help provide reliable detection of conditions causing inaccurate tracking of an object. Certain embodiments of the VT 400 may also facilitate a tracking system that operates with SNR-limited tracking accuracy (e.g., reduced systematic errors).

A variety of systems, methods, and objects may be used for distortion detection, compensation, and tolerance in accordance with certain embodiments of the present invention. Several examples are described below for illustrative purposes.

In an embodiment, if a distorting object (a distorter) is fixed with respect to an EM transmitter, distortion from the distorter may be mapped using a variety of methods. For each transmitter coil in a transmitter, a normal component of a magnetic field may be measured on a boundary surface surrounding the distorter. An undistorted field value may be subtracted from the measurement to determine the field from the distorter on the boundary. In an embodiment, the distorter's field outside the boundary is calculated using a Laplace equation and finite-element analysis. In another embodiment, the distorter is modeled as an array of dipoles of various positions, orientations, and/or strengths, for example, inside the boundary. The positions, orientations, and/or strengths of the dipoles are adjusted to fit the field at the boundary. Then, a transmitter dipole is added to the distorter dipole array. The dipoles form an analytical model for the transmitter and distorter outside the measured boundary. Using the model, a magnetic field, a magnitude-squared of the field, and a field gradient may be calculated at any point outside the boundary.

For transmitter coil field magnitudes, a set of eight functions, for example, may be calculated from a trio of transmitter coil field magnitudes squared. For example, one function may be determined for each combination of x, y, and z component signs. Table look-up interpolations and/or Raab's signal-matrix-sign function may be used to determine the functions, for example. In an embodiment, the eight functions are equal in an absence of distortion. An inversion of the functions may produce a distortion mapping. Additionally, the functions may be rotated to produce a desired mapping. A least-squares best-fit solution for distortion may be calculated using the field and gradient modules.

Alternatively, a signal matrix may be measured from the distorter's field. Then, transmitter coil field magnitudes are calculated. Approximate positions for undistorted transmitter coils are determined (using Raab's algorithm, for example). Signs for the transmitter coils are chosen and a candidate solution selected. If the solution is close to an axis, the matrix functions may be rotated away from the axis. A solution may be obtained. Signs are chosen. The matrix is then rotated back to the original position. The solution represents an approximate distortionless solution. Then, a least-squares best-fit solution is calculated using calculated undistorted transmitter dipoles. The solution includes distortion error. A second least-squares best-fit solution is calculated using field and gradient models including distortion. Repeating a distorted least-squares fit may improve the result due to second-order effects.

In an embodiment, an ISCA distortion mapping process may be used to model a distorter as an array of dipoles. The distortion-generating dipoles are excited by a dipole field from a transmitter. A distorter dipole gain is a ratio of a distorter dipole moment to an undistorted transmitter field at a location of the distorter dipole. The distorter dipole is in a direction to oppose the transmitter field. The distorter dipole, however, may not be parallel to the transmitter field. Thus, distorter gain may be a matrix or a tensor, for example, because the distorter response may differ for undistorted transmitter field components in x, y, and z directions. In an embodiment, the gain matrix or tensor is independent of the transmitter fields. If the transmitter is moved, the distortion gain remains the same. The distortion field may be recalculated using new values of transmitter fields at the locations of the distorters. Thus, distortion compensation may be tuneable by a few parameters to correct for a moved transmitter.

For example, a receiver may be mounted on a debrider blade. The blade mounts into a debrider housing on axis with an uncontrolled roll angle. The housing causes distortion. The housing is unsymmetrical, so the distortion varies with a roll of the housing with respect to the receiver. A distortion map with a tracking-time adjustable parameter, such as a roll value of the map, may accommodate the distortion from the debrider housing. Acquiring additional data points allows calculation of the map roll value to accommodate distorters fixed with respect to either the transmitter or receiver or elsewhere. If more than one distorter is present, iterative analyses may account for effects of the distorters on each other. That is, each distorter may be affected by a field due to the transmitter and a field due to another distorter, for example.

In an embodiment, distortion of a conducting ring may be modeled in the distortion handling system 100. A position and orientation of the ring are determined with respect to a transmitter. A shape of the ring is also determined. A cross-section of the conductor is small compared to the size of the ring. Thus, the conductor may be treated as infinitesimal when calculating a mutual inductance between the ring and a transmitter coil. Additionally, self-inductance of the conductor may be ignored when calculating a self-inductance of the ring. Then, the self-inductance of the ring may be calculated from the shape of the ring. The mutual inductance between the ring and the transmitter coil may be calculated from Feynman's mutual inductance double integral, for example. A transmitter model for the transmitter coil may be used to determine a magnetic field per coil current, in teslas per ampere, for a certain location (x,y,z). A field model for the distorter ring may then be calculated from a current value and a field per current value.

In an embodiment, a transmitter coil board including a larger number of small spiral coils may be used for distortion mapping of magnetic fields. Coils in the board are driven one at a time by a driver board. In an embodiment, one frequency drives the time-multiplexed coils on the coil board. A component side of the board is directed toward the inside of a volume. A solder side of the board faces the outside of the volume. A device under test is positioned inside the volume. The component side of the board electrostatically shields the solder side of the board from the device under test and provides a return path for a driver current.

Each coil on the board is connected to a switch. A coil and corresponding switch are connected in series. Unselected coils are at an alternating current ground. A selected coil's current flows to a current measuring device. A switch may be an optocoupler, a diode or series of diodes, and/or a transistor, for example. A Y line on the component side is driven by an alternating current source. Each Y line is electrostatically shielded by a grounded track on the component side. An X line is located on the component side and is at an alternating current ground or a virtual ground. The X line is connected to the current measuring device. A coil is connected in series with a switch at the intersection of the X and Y lines. In an embodiment, the board includes a plurality of coils and switches connected in series at the intersections of a plurality of X and Y lines. The coil board or driver board may include time-multiplexing logic to power the coils. The time-multiplexed coils and current measuring device are used to create a distortion mapping of magnetic fields around the device under test.

In an embodiment, a fluoroscopic imaging system uses a pair of ISCA receivers mounted on a fluoro camera calibration fixture. The fluoro camera calibration fixture may be mounted on an image intensifier tube, for example. The image intensifier includes various metals and distorts a magnetic field from an electromagnetic tracker. A robotic system may be used to map the distortion and generate a tracker correction table. The table may then be used to correct tracking errors due to distortion. Different image intensifiers of a same model exhibit slightly different magnetic field distortions. Thus, each intensifier may employ a different distortion correction table.

A distorting can may be used in the camera calibration fixture. In an embodiment, the can includes a single piece of highly conducting metal, such as copper, silver, or aluminum. The can surrounds the image intensifier as much as possible, consistent with mechanical constraints and allowing for an x-ray transparent region in front of the image intensifier. The can also distorts the magnetic field of the tracker. Thus, the can's distortion is mapped and corrected with a generated tracker correction table. In an embodiment, a thickness of the can metal is more than a skin depth of the metal such that the magnetic field does not appreciably penetrate the metal and the volume behind the metal. The can shields most of the magnetic field from the image intensifier so that the intensifier has little field to distort. Thus, using the can, changing the image intensifier has no appreciable effect on the field distortion. One distortion correction table may be used for all image intensifiers of a given model. In another embodiment, one distortion correction table may be used for image intensifiers of different models.

Additionally, a fluoroscopic or other x-ray system may include an x-ray scatter filter. In an embodiment, the x-ray scatter filter includes alternating layers of lead and x-ray transparent material, such as plastic or aluminum. An x-ray aperture may be provided that conducts electricity and thus blocks the magnetic field. A filter may fill the x-ray aperture of a conductive sheath covering the image intensifier of a fluoroscope. The sheath keeps a tracker magnetic field away from the image intensifier so that replacing the image intensifier has minimal effect on the tracker.

In an embodiment, a passive conductive ring may be placed close to a receiver in a fluoroscopy system. The passive ring may be placed asymmetrically, for example, with respect to an ISCA transmitter. The passive conductive ring disambiguates a hemisphere of the tracker system without a large wound coil. Alternatively, a ferromagnetic rod may be placed with one end close to the ISCA transmitter to disambiguate the hemisphere. Additionally, reciprocity permits the ring or rod to be placed close to a receiver instead of the transmitter to disambiguate.

In an embodiment, an existing ISCA seed routine may be mapped to allow calculation of a seed for distorters fixed with respect to a transmitter in the system 100. For example, squares of position components (x², y², z²) are calculated from squares of sums of three receiver coil mutual inductances for each transmitter coil. In an embodiment, the inductance squares are independent of receiver orientation. If a distorter is fixed with respect to the transmitter, a map may be calculated to replace an analytic dipole solution for the distorter. For the distorter, the inductance squares may still be independent of receiver orientation. Additionally, reciprocity allows the receiver and transmitter to be swapped and position and orientation information determined for a distorter fixed with respect to the receiver.

In an embodiment, after magnetic field mapping data has been determined, several data reduction methods may be used. For example, Laplace's equation and finite element methods may be used on boundary points of the field map. Interior points may be used to check accuracy. A distorter model may then be expressed in several forms. The model may be a lookup table with an interpolation method, functional fits, and/or a parameterized sum of basis functions, for example. Basis functions may be chosen for mathematical convenience and/or for electromagnetic compatibility. For example, basis functions may be created relation to field due to distortion rings, rods, and/or sheets of parameterized sizes. Distorter models may be expressed in terms of calculationally convenient functions. Other basis functions may include dipoles of various strengths and orientations and Green's functions, for example. In an embodiment, an electromagnetically sensible basis results in parameter values providing solutions that satisfy Laplace's equation. Additionally, “good” models with relatively few parameters are produced. Families of maps may be produced from the models. Families of maps may provide distortion mapping with fewer data points.

In an embodiment, a simple dipole model may be created for a small conducting-loop distorter. The distorter is fixed with respect to an electromagnetic transmitter. The distorter is assumed to be perfectly conducting. A field from the transmitter creates a flux through the loop. The loop includes eddy currents that force a total flux through the loop to be zero. From measurements of the eddy currents, a distortion field due to the loop may be calculated. For a large loop, a distortion field may be calculated using integrals. For a small loop, the loop may be approximated by a dipole, and the field due to the eddy currents may be determined. For example, an area of the loop, eddy currents in the loop, and flux through the loop may be used to determine a distortion field from the loop. In an embodiment, the distortion field may cause changes in transmitter currents, but the changes are measured and corrected by tracker electronics.

In another embodiment, a simple dipole model may be created for a small conducting-object distorter. The distorter is fixed with respect to an electromagnetic transmitter. The distorter is assumed to be perfectly conducting and to not permit flux in the interior of the distorter. A distortion field of the object distorter may be approximated by identifying a large cross-section of the object in a plane normal to an incident transmitter field. The cross-sectional area may be used to calculate a distorting field as described above in relation to the small conducting-loop distorter. In an embodiment, distorter effective areas are different for different-direction incident transmitter fields.

In another embodiment, a small infinite-permeability non-conducting ferrite object near a transmitter may be analyzed. The object is preferably fixed with respect to the transmitter. The object doubles or triples, for example, an incident flux in the interior of the object. A distortion field of the object may be approximated as described above with respect to a small conducting-object distorter. The distortion field may then be multiplied by a flux factor, such as −2 or −3, to account for the incident flux in the interior of the object.

A variety of instruments may be tested in the distortion handling system 100 and/or with the VT 400 to determine distortion effects on the tracking system and calibrate the tracking system based on the distortion effects. For example, a pneumatic or electric drill may be examined to determine an effect of the drill on positional and orientational accuracy of the tracking system. Additionally, the distortion handling system 100 may be used to estimate an appropriate attachment distance between an EM receiver pack and the drill. An appropriate receiver attachment distance may improve tracking of an instrument 110, such as the drill. Use of an FID may improve detection of errant tracking due to magnetic field distortion by an instrument 110, such as a drill.

In order to examine the drill, a sensor is placed apart from the transmitter and parallel to an x-axis of a tracking coordinate system. The transmitter and receiver pack are attached to a board. Position and orientation data are collected without the drill present to obtain calibration or reference data. Then, the drill is placed at a plurality of distances from the receiver pack. The orientation of the receiver pack to a top surface of the drill is kept parallel for the plurality of distances. Position and orientation is collected for the plurality of distances. Tracker goodness of fit (GOF) data may also be recorded at the plurality of locations. Next, position data may be computed for a tip of the drill, for example. Error in the tip position is determined using a plurality of test conditions. FID data is computed using a navigation algorithm and threshold. Since both the orientation of the receiver pack and the tip offset were parallel to the x-axis of the tracking coordinate system, a theoretical drill guide tube trajectory error may be estimated by taking an x component of the tip error.

Thus, certain embodiments of the present invention provide a tracking system and method that are robust and tolerant of incidental distortions. Certain embodiments provide ergonomic and efficient integration of EM position sensors with surgical tools or other instruments. Certain embodiments provide reliable detection of conditions causing inaccurate tracking. Certain embodiments minimize systematic errors with signal-to-noise ratio limited tracking accuracy. Tracking errors due to metal distortion may be analyzed and compensated for using an electromagnetic tracking system and distortion handler.

Certain embodiments provide a virtual tracking system and simulation environment used to measure distortion and other effects and adjust configuration of an instrument being tracked, an instrument guide, and/or a tracking system. Certain embodiments provide a system and method for minimizing distortion in a tracking system. Certain embodiments provide a system and method for developing and testing distortion-tolerant instruments and tracking systems.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. An improved electromagnetic tracking system, the system comprising:
a transmitter for transmitting a signal;
a receiver for receiving the signal from the transmitter, said transmitter and receiver for use in tracking an object; and
a distortion handling system for analyzing a distortion characteristic of the object.

2. The system of claim 1, wherein the distortion handling system analyzes the signal received by the receiver to determine tracking accuracy.

3. The system of claim 1, wherein the distortion handling system comprises a virtual electromagnetic tracker for simulating an effect of the distortion characteristic on tracking.

4. The system of claim 1, wherein the distortion handling system adjusts a tracking behavior of the object.

5. The system of claim 1, wherein the distortion characteristic comprises a magnetic field.

6. A distortion handling system, the system comprising:
a tracking analysis unit for analyzing a tracking behavior of an instrument; and
a tracking modification unit for compensating for the tracking behavior of the instrument.

7. The system of claim 6, wherein the tracking analysis unit further tests an adjusted tracking behavior of the instrument.

8. The system of claim 6, wherein the tracking modification unit compensates for the tracking behavior of the instrument by adjusting the tracking behavior of the instrument.

9. The system of claim 6, wherein the tracking modification unit compensates for the tracking behavior of the instrument by adjusting a tracking system for tracking the instrument.

10. The system of claim 6, wherein the tracking analysis unit generates at least one of a map and a model of a distortion characteristic of the instrument.

11. The system of claim 10, wherein the distortion characteristic comprises a magnetic field.

12. The system of claim 6, wherein the distortion handling system comprises a computer simulated distortion handling system.

13. A virtual tracking system, the system comprising:
an object simulation module for simulating an object to be tracked; and
a simulation toolset for analyzing at least one distortion characteristic of the object, the simulation toolset capable of generating distortion information for the object based on the at least one distortion characteristic.

14. The system of claim 13, wherein the simulation toolset further comprises an accuracy module for determining accuracy of simulated tracking of the object.

15. The system of claim 13, wherein the simulation toolset further comprises a distortion detection module for determining an impact of distortion from the object on tracking accuracy.

16. The system of claim 13, wherein the simulation toolset further comprises a distortion modeling module for generating an electromagnetic model for evaluating a distorting effect of the object on a tracker environment.

17. The system of claim 13, wherein the simulation toolset further comprises a distortion compensation module for improving distortion tolerance of an electromagnetic tracker.

18. A method for distortion analysis in an electromagnetic tracking system, the method comprising:
measuring a tracking behavior of an object; and
analyzing the tracking behavior of the object to determine a distortion effect.

19. The method of claim 18, wherein the analyzing step further comprises generating at least one of a field map and a model to analyze the tracking behavior of the object to determine the distortion effect.

20. The method of claim 18, further comprising simulating tracking of the object to determine the distortion effect.

21. The method of claim 18, further comprising adjusting the tracking behavior of the instrument to reduce the distortion effect.

22. The method of claim 21, further comprising testing the instrument to verify a reduced distortion effect.

23. The method of claim 18, further comprising adjusting an electromagnetic tracker to compensate for the distortion effect.

24. The method of claim 23, further comprising testing the instrument to verify a reduced distortion effect.

25. The method of claim 18, further comprising modeling electromagnetic fields in an electromagnetic tracking environment to analyze the distortion effect.

26. The method of claim 18, further comprising improving distortion tolerance in an electromagnetic tracking system based on the distortion effect.
